(19) United States
(12) Patent Application Publication
(10) Pub. No.: US 2010/0104663 A1
(43) Pub. Date: Apr. 29, 2010

(54) REHYDRATION COMPOSITION FOR PREPARING A SOLUTE BY RECONSTITUTION IN WATER

(75) Inventor: Daniel Collin, Saint Malo (FR)

Correspondence Address: HAMRE, SCHUMANN, MUELLER & LARSON, P.C., P.O. BOX 2902, MINNEAPOLIS, MN, 55402-0902, US

(73) Assignee: LABORATOIRES ADITEC, Dinard (FR)

(21) Appl. No.: 12/529,804

(22) PCT Filed: Mar. 6, 2008
(86) PCT No.: PCT/FR2008/050377

§ 371 Date: Sep. 3, 2009

(30) Foreign Application Priority Data
Mar. 6, 2007 (FR) 0753665

(51) Int. Cl. A61K 33/14; A61K 33/00

(52) U.S. Cl. 424/678; 424/722

(57) ABSTRACT

The present invention relates to a rehydration composition intended for the preparation of a solution by reconstitution in water, preferably slightly mineralized water.

According to the invention, this composition comprises the following constituents in the specific proportions mentioned in claim 1: sodium; potassium; chloride; glucose-providing carbohydrate source; at least one water-soluble amino compound chosen from amino acids, peptides and polypeptides; at least one vitamin chosen from: vitamin C; vitamin B1; vitamin B2; vitamin B3; vitamin B6; vitamin B8; vitamin B9; vitamin B12; said solution having an osmolarity between 150 and 200 mosmol/l.

This composition is particularly suitable for the rehydration of elderly people.

The subject of the present invention is a novel rehydration composition intended for the preparation, by reconstitution in water, of a solution suitable for the prevention and treatment of dehydration resulting from intense heat.

It is known that the human body consists of about 70% water, of which about two thirds are found inside the cells. Water is therefore an essential constituent for the proper functioning of the human body.

Dehydration occurs when the normal water content of the body is reduced, for example because of insufficient intake of water or a loss of a large quantity of body fluid which is not replaced. It causes in particular a modification of the water-electrolyte balance on which the proper functioning of most cells depends, and which can cause particularly serious consequences.

The causes of dehydration may vary, with different consequences at the intracellular or extracellular level and in terms of loss of water and salt.

Dehydration results mainly from digestive disorders (persistent diarrhea or vomiting associated with gastroenteritis) or from excessive perspiration due to fever or intense heat.

The best means of treating dehydration is the taking of fluid and numerous oral rehydration solutions have been recommended to this effect.

Several oral rehydration solutions intended for the treatment of unweaned babies suffering from acute diarrhea are currently marketed in industrialized countries.

On the other hand, there is up until now no rehydrating composition specifically intended for the treatment of heat-related dehydration states on the market.

However, the dehydration resulting from heat waves (scorching heat) can have serious consequences which may result in death, the risks being particularly high in babies, unweaned babies and elderly people.

A real need therefore exists for a rehydrating composition specifically suitable for the prevention and treatment of dehydration resulting from intense heat.

In this context, the aim of the present invention is to satisfy this need by providing a novel composition intended for the preparation of an oral rehydration solution suitable in particular for the needs of elderly people.

Accordingly, the object of the present invention is a rehydration composition intended for the preparation of a solution by reconstitution in water, preferably slightly mineralized water, characterized in that it comprises, expressed as quantity relative to one liter of final solution:
35 to 75 mmol per liter of sodium
5 to 15 mmol per liter of potassium
11 to 50 mmol per liter of chloride
1 to 5 grams per liter of a source of glucose-providing carbohydrate
0.1 to 5 grams per liter of at least one water-soluble amino compound chosen from amino acids, peptides and polypeptides;
at least one vitamin chosen from:
   vitamin C, in a quantity of 25 mg/l to 1 g/l
   vitamin B1, in a quantity of 0.1 mg/l to 2.6 mg/l
   vitamin B2, in a quantity of 0.2 mg/l to 3.2 mg/l
   vitamin B3, in a quantity of 1.5 mg/l to 28 mg/l
   vitamin B6, in a quantity of 0.15 mg/l to 4.4 mg/l
   vitamin B8, in a quantity of 3 μg/l to 120 μg/l
   vitamin B9, in a quantity of 30 μg/l to 2000 μg/l
   vitamin B12, in a quantity of 0.5 μg/l to 20 μg/l
      said solution having an osmolarity between 150 and 200 mosmol/l.

Among the compositions which satisfy the characteristics mentioned above, a specific example consists in particular of compositions comprising from 35 to 55 mmol of sodium and from 5 to 8 mmol of potassium per liter of final solution.

According to the invention, a particularly preferred rehydration composition comprises (expressed as quantity relative to one liter of final solution):
35 to 65 mmol per liter of sodium
5 to 15 mmol per liter of potassium
15 to 45 mmol per liter of chloride
2 to 5 grams per liter of a source of glucose-providing carbohydrate
1 to 4 grams per liter of at least one water-soluble amino compound chosen from amino acids, peptides and polypeptides;
at least one vitamin chosen from:
   vitamin C, in a quantity of 25 mg/l to 1 g/l
   vitamin B1, in a quantity of 0.1 mg/l to 2 mg/l
   vitamin B2, in a quantity of 0.2 mg/l to 3.2 mg/l
   vitamin B3, in a quantity of 1.5 mg/l to 28 mg/l
   vitamin B6, in a quantity of 0.2 mg/l to 3.3 mg/l
   vitamin B8, in a quantity of 3 μg/l to 120 μg/l
   vitamin B9, in a quantity of 100 μg/l to 1000 μg/l
   vitamin B12, in a quantity of 1 μg/l to 15 μg/l
      said solution having an osmolarity between 150 and 200 mosmol/l.

Another example of a composition according to the invention comprises (expressed as quantity relative to one liter of final solution):
40 to 50 mmol per liter of sodium
5 to 8 mmol per liter of potassium
20 to 40 mmol per liter of chloride
2 to 4 grams per liter of a source of glucose
0.5 to 3 grams per liter of at least one water-soluble amino compound chosen from amino acids, peptides and polypeptides;
at least one vitamin chosen from:
   vitamin C, in a quantity of 50 mg/l to 0.5 g/l
   vitamin B1, in a quantity of 0.2 mg/l to 1.3 mg/l
   vitamin B2, in a quantity of 0.4 mg/l to 1.6 mg/l
   vitamin B3, in a quantity of 3 mg/l to 15 mg/l
   vitamin B6, in a quantity of 0.30 mg/l a 2.2 mg/l
   vitamin B8, in a quantity of 6 μg/l to 60 μg/l
   vitamin B9, in a quantity of 400 μg/l to 700 μg/l
   vitamin B12, in a quantity of 1 μg/l to 10 μg/l
      said solution having an osmolarity between 150 and 200 mosmol/l.

The composition according to the invention is particularly novel since it makes it possible to compensate for the perspiration losses which are in particular induced by heat, while providing a supply of mineral, in particular sodium, salts, avoiding any water-electrolyte imbalance; the hydration being enhanced not only by a supply of glucose, but also by a supply of judiciously selected amino acid(s) and vitamin(s).

The rehydration composition according to the invention contains a sodium source, a potassium source and a chloride source in quantities such that the concentration of each of these elements in the final solution is as indicated above.

These elements are provided in particular by mineral or organic salts such as for example sodium chloride, sodium bicarbonate, sodium acetate, sodium hydrogencarbonate, sodium carbonate, potassium chloride, potassium phosphate.

These elements are mainly intended to compensate for the loss of minerals accompanying sweat production.

These elements may also be provided by a spray-dried product of sea water. Such a spray-dried product of sea water constitutes a source of magnesium, an essential metal used as cofactor in numerous enzymatic reactions. Since sea water is relatively low in potassium, a supplement of potassium source, for example in chloride form, will have to be added to the spray-dried product of sea water.

The rehydration composition according to the invention also comprises a source of glucose in a concentration of 1 to 5 grams per liter of final solution.

The glucose enhances the absorption of sodium by the phenomenon of sodium-glucose co-transport.

The relatively low amount of carbohydrates is particularly advantageous for the rehydration of elderly people, since the presence of glucose at a higher level could be deleterious in particular in situations of hyperglycemia, as well as in people suffering from insulin-dependent diabetes.

According to a particular characteristic of the invention, the glucose source is chosen from glucose, dextrose, disaccharides or polysaccharides comprising at least one glucose subunit, such as in particular sucrose, lactose, starch, maltose, cellobiose, trehalose and mixtures thereof.

According to a preferred embodiment, the glucose source is chosen from sucrose, lactose and a mixture of lactose and dextrose.

The rehydration composition according to the invention also comprises at least one water-soluble amino compound chosen from amino acids, peptides and polypeptides, in a quantity of 0.1 to 5 g per liter of final solution.

This amino compound also promotes the absorption of sodium by a phenomenon of sodium-amino acid co-transport.

In the present description, the expression “peptide” is understood to mean any chain comprising less than 20 amino acids and the expression “polypeptide” is understood to mean any chain containing from 20 to 100 amino acids.

These amino compounds will be preferably chosen in order to satisfy the specific needs of elderly people.

According to a particular characteristic, the water-soluble amino compound is chosen from methionine and glutamine.

Accordingly, glutamine constitutes a preferred amino acid in the context of the present invention since it acts:
as a structuring agent by playing a direct role in the loss of muscle mass linked to aging (sarcopenia);
as a glutathione precursor by having an indirect antioxidant role since glutathione is the most important antioxidant enzyme in the body;
as an immune agent, thus directly involved in immunity by stimulating the secretion of IgA, an immunoglobulin present at the level of the digestive and respiratory membranes acting as the first immune system barrier in the body.

Another particularly preferred amino acid in the context of the present invention is methionine which, because of its properties as precursor of creatine, choline and carnitine, will have a role in maintaining the muscle mass.

Finally, glycine also constitutes an amino acid which may be advantageously used for the implementation of the present invention.

The rehydration composition according to the invention also comprises at least one vitamin chosen from vitamin C (ascorbic acid), vitamin B1 (thiamine or preferably thiamine triphosphate), vitamin B2 (riboflavin), vitamin B3 (niacin), vitamin B6 (pyridoxine or pyridoxamine or pyridol), vitamin B8 (biotin), vitamin B9 (folic acid) and vitamin B12 (cobalamine) in the quantities mentioned above.

According to a particular embodiment, the rehydration composition according to the invention comprises, for one liter of final solution, the following mixture of vitamins:
vitamin C: from 50 mg/l to 0.5 g/l, preferably from 175 mg/l to 225 mg/l;
vitamin B1: from 0.2 mg/l to 1.3 mg/l, preferably from 1.15 to 1.3 mg/l;
vitamin B6: from 0.3 mg/l to 2.2 mg/l, preferably from 2 to 2.2 mg/l.

According to another particularly preferred embodiment, the rehydration composition according to the invention comprises, for one liter of final solution, the following mixture of vitamins:
vitamin B1: from 0.1 mg/l to 2 mg/l, preferably from 0.5 to 1.8 mg/l;
vitamin B6: from 0.2 mg/l to 3.3 mg/l, preferably from 1 to 3 mg/l;
vitamin B9: from 100 μg/l to 1000 μg/l, preferably from 400 μg/l to 700 μg/l;
vitamin B12: from 1 μg/l to 15 μg/l, preferably from 1 μg/l to 10 μg/l.

These vitamins will be preferably chosen to satisfy the specific needs of elderly people.

The rehydration composition according to the invention may be provided in a variety of forms, preferably in a dry form, in particular in tablet, powdered or granule form. It may also be prepared in the form of a concentrated liquid.

According to a particular embodiment, this composition will be provided in effervescent form. To this end, the composition will comprise an effervescent system capable of producing carbon dioxide gas in the presence of water consisting for example of an alkali metal carbonate and an organic acid.

An alkali metal carbonate which may be used in the context of the invention is for example sodium bicarbonate, potassium bicarbonate, calcium carbonate, sodium carbonate, potassium carbonate, sodium hydrogen carbonate, potassium hydrogen carbonate and mixtures of these compounds.

The presence of carbonate advantageously makes it possible to also combat metabolic acidosis following dehydration.

An organic acid which can be used in the context of the present invention is for example citric acid, fumaric add, adipic acid, tartaric acid and mixtures of these compounds

According to a currently preferred embodiment, the effervescent system of a rehydration composition according to the invention will consist of a mixture of sodium bicarbonate and citric acid.

The rehydration composition according to the invention may be prepared in a conventional manner by mixing the constituents, preferably in powdered form or by granulation in the form of granules which may be packaged directly for example in a sachet or alternatively provided as tablets after a traditional compression step.

More specifically, the rehydration composition according to the invention may be prepared according to a manufacturing principle involving a mixing process using a LODIGE-type mixer or a wet or dry granulation system which may use a planetary-type mixer. In the case of wet granulation, it will also be necessary to carry out a drying step in an oven or on a fluidized air bed.

When the product obtained is a powder mixture, it will be packaged in a sachet by means of a VOLPAK-type horizontal packet-filling machine or by a vertical packet-filling machine.

For the production of a tablet, a compression may be carried out by means of a so-called alternating or rotary system.

The rehydration composition which has just been described is intended for the preparation, by reconstitution in water that is preferably slightly mineralized, of a solution having an osmolarity between 150 and 200 mosmol/l. Persons skilled in the art will know how to adjust the composition and the reconstitution volume in order to obtain an osmolarity within this range.

This solution is advantageously provided in the form of a liquid solution.

It may also be provided in a pasty form. To this end, the composition will contain an effective quantity of a gelling agent. Such an agent may be for example an alginate, a carrageenan, modified cornstarch or alternatively a pectin, in particular apple pectin.

In general, the rehydration composition according to the invention will be provided in the form of unit doses to be reconstituted in a glass of water with an average volume equal to 20 centiliters.

Such unit doses may be taken at the rate of 3 to 10 times per day and preferably 3 to 5 times per day according to the state of dehydration.

The invention will now be illustrated by various examples of compositions using the principles recalled above.

### EXAMPLE 1

Composition according to the invention provided in effervescent solid form intended to be used on an outpatient basis and to be easily dissolved in a glass of water with an average volume of 20 centiliters.

| | | |
|---|---|---|
| Sodium | 0.481 g to 0.689 g | preferably 0.51 g to 0.59 g |
| bicarbonate: | | |
| NaCl: | 0.074 g to 0.164 g | preferably 0.082 g to 0.089 g |
| KCl: | 0.0745 g to 0.1192 g | preferably 0.13 g to 0.17 g |
| Citric acid: | 0.2 g to 0.4 g | preferably 0.23 g to 0.3 g |
| Vitamin C: | 0.01 g to 0.1 g | preferably 0.035 g to 0.045 g |
| Vitamin B1: | 0.04 mg to 0.26 mg | preferably 0.23 mg to 0.26 mg |
| Vitamin B6: | 0.06 mg to 0.44 mg | preferably 0.4 mg to 0.44 mg |
| Vitamin B12: | 0.6 microgram | |
| Glycine: | 0.01 g to 0.5 g | preferably 0.04 g to 0.07 g |
| Methionine: | 0.01 g to 0.5 g | preferably 0.04 g to 0.07 g |
| Sucrose: | 0.2 g to 1 g | preferably 0.05 g to 0.07 g |

### EXAMPLE 2

Composition according to the invention provided in solid form intended to be used on an outpatient basis and to be easily dissolved in a glass of water with an average volume of 20 centiliters.

| | |
|---|---|
| NaCl: | 0.123 g to 0.243 g |
| KCl: | 0.0745 g to 0.1192 g |
| Trisodium citrate, 2H₂O: | 0.48 g to 0.67 g |
| Vitamin C: | 0.01 g to 0.1 g |
| Vitamin B1: | 0.04 mg to 0.26 mg |
| Vitamin B6: | 0.06 mg to 0.44 mg |
| Vitamin B12: | 0.6 microgram |
| Glycine: | 0.01 g to 0.5 g |
| Methionine: | 0.01 g to 0.5 g |
| Sucrose: | 0.2 g to 1 g |

### EXAMPLE 3

Composition according to the invention provided in the form of thickened water intended for the rehydration of people sensitive to false passages.

The quantities mentioned are given for 100 g mass/mass

| | |
|---|---|
| NaCl: | 0.062 g to 0.121 g |
| KCl: | 0.038 g to 0.059 g |
| Trisodium citrate, 2H₂O: | 0.24 g to 0.335 g |
| Vitamin C: | 0.005 g to 0.05 g |
| Methionine: | 0.005 g to 0.25 g |
| Glycine: | 0.005 g to 0.25 g |
| Sucrose: | 0.1 g to 0.5 g |
| Apple pectin: | 2 g to 4 g |
| Water: | qs 100 g |

### EXAMPLE 4

Composition according to the invention provided in the form of thickened water intended for the rehydration of people sensitive to false passages.

The quantities mentioned are given for 100 g mass/mass

| | |
|---|---|
| NaCl: | 0.062 g to 0.121 g |
| KCl: | 0.038 g to 0.059 g |
| Trisodium citrate, 2H₂O: | 0.24 g to 0.335 g |
| Vitamin C: | 0.005 g to 0.05 g |
| Methionine: | 0.005 g to 0.25 g |
| Glycine: | 0.005 g to 0.25 g |
| Cornstarch: | 1 g to 2 g |
| Water: | qs 100 g |

### EXAMPLE 5

Preferred formulation provided in effervescent solid form to be reconstituted in 20 centiliters of water.

| | | |
|---|---|---|
| Sodium bicarbonate: | 0.55 | g |
| NaCl: | 85 | mg |
| KCl: | 105 | mg |
| Citric acid: | 0.25 | g |
| Vitamin C: | 39.62 | mg |
| Vitamin B1: | 0.259 | mg |
| Vitamin B6: | 0.439 | mg |
| Vitamin B12: | 0.54 | microgram |
| Glycine: | 69 | mg |
| Methionine: | 69 | mg |
| Sucrose: | 630 | mg |
| PEG: | 100 | mg |
| PVP: | 100 | mg |

### EXAMPLE 6

Currently preferred formulation provided in effervescent solid form to be reconstituted in 20 centiliters of water (quantities indicated for pure products).

| | | |
|---|---|---|
| Sodium bicarbonate: | 624.316 | mg |
| NaCl: | 317.400 | mg |
| KCl: | 212.010 | mg |
| Citric acid: | 356.696 | mg |
| Vitamin B1: | 0.32 | mg |
| Vitamin B6: | 0.59 | mg |
| Vitamin B9: | 133 | micrograms |
| Vitamin B12: | 1 | microgram |
| Glycine: | 126 | mg |
| Lactose: | 368.280 | mg |
| Dextrose: | 570 | mg |
| Gum acacia (fiber gum): | 408 | mg |

After reconstitution, the osmolarity of the solution obtained is 178 mosmol/l.

1. A rehydration composition intended for the preparation of a solution by reconstitution in water, which comprises, expressed as quantity relative to one liter of final solution:
35 to 75 mmol per liter of sodium
5 to 15 mmol per liter of potassium
11 to 50 mmol per liter of chloride
1 to 5 grams per liter of a source of glucose-providing carbohydrate
0.1 to 5 grams per liter of at least one water-soluble amino compound selected from the group consisting of amino acids, peptides and polypeptides;
at least one vitamin selected from the group consisting of:
vitamin C, in a quantity of 25 mg/l to 1 g/l
vitamin B1, in a quantity of 0.1 mg/l to 2.6 mg/l
vitamin B2, in a quantity of 0.2 mg/l to 3.2 mg/l
vitamin B3, in a quantity of 1.5 mg/l to 28 mg/l
vitamin B6, in a quantity of 0.15 mg/l to 4.4 mg/l
vitamin B8, in a quantity of 3 μg/l to 120 μg/l
vitamin B9, in a quantity of 30 μg/l to 2000 μg/l
vitamin B12, in a quantity of 0.5 μg/l to 20 μg/l said solution having an osmolarity comprised between 150 and 200 mosmol/l.

2. The composition as claimed in claim 1, which comprises, expressed as quantity relative to one liter of final solution:
35 to 65 mmol per liter of sodium
5 to 15 mmol per liter of potassium
15 to 45 mmol per liter of chloride
2 to 5 grams per liter of a source of glucose-providing carbohydrate
1 to 4 grams per liter of at least one water-soluble amino compound selected from the group consisting of amino acids, peptides and polypeptides;
at least one vitamin selected from the group consisting of:
vitamin C, in a quantity of 25 mg/l to 1 g/l
vitamin B1, in a quantity of 0.1 mg/l to 2 mg/l
vitamin B2, in a quantity of 0.2 mg/l to 3.2 mg/l
vitamin B3, in a quantity of 1.5 mg/l to 28 mg/l
vitamin B6, in a quantity of 0.2 mg/l to 3.3 mg/l
vitamin B8, in a quantity of 3 μg/l to 120 μg/l
vitamin B9, in a quantity of 100 μg/l to 1000 μg/l
vitamin B12, in a quantity of 1 μg/l to 15 μg/l.

3. The composition as claimed in claim 1, characterized in that it which comprises, expressed as quantity relative to one liter of final solution:
40 to 50 mmol per liter of sodium
5 to 8 mmol per liter of potassium
20 to 40 mmol per liter of chloride
2 to 4 grams per liter of a source of glucose
0.5 to 3 grams per liter of at least one water-soluble amino compound chosen selected from the group consisting of amino acids, peptides and polypeptides;
at least one vitamin chosen from:
vitamin C, in a quantity of 50 mg/l to 0.5 g/l
vitamin B1, in a quantity of 0.2 mg/l to 1.3 mg/l
vitamin B2, in a quantity of 0.4 mg/l to 1.6 mg/l
vitamin B3, in a quantity of 3 mg/l to 15 mg/l
vitamin B6, in a quantity of 0.30 mg/l a 2.2 mg/l
vitamin B8, in a quantity of 6 μg/l to 60 μg/l
vitamin B9, in a quantity of 400 μg/l to 700 μg/l
vitamin B12, in a quantity of 1 μg/l to 10 μg/l.

4. The composition as claimed in claim 1 wherein the glucose source is selected from the group consisting of glucose, dextrose, disaccharides and polysaccharides comprising at least one glucose subunit.

5. The composition as claimed in claim 1 wherein the water-soluble amino compound is selected from the group consisting of methionine, glutamine and glycine.

6. The composition as claimed in claim 1 wherein the sodium is provided by a compound selected from the group consisting of sodium chloride, sodium bicarbonate, sodium acetate, sodium hydrogen carbonate and sodium carbonate.

7. The composition as claimed in claim 1 which is provided in the form of a tablet, a powder or a granule.

8. The composition as claimed in claim 7, which is provided in effervescent form.

9. The composition as claimed in claim 1 which is provided in the form of a unit dose to be reconstituted in 20 cl of water and which comprises:

| | | |
|---|---|---|
| Sodium bicarbonate: | 0.55 | g |
| NaCl: | 85 | mg |
| KCl: | 105 | mg |
| Citric acid: | 0.25 | g |
| Vitamin C: | 39.62 | mg |
| Vitamin B1: | 0.259 | mg |
| Vitamin B6: | 0.439 | mg |
| Vitamin B12: | 0.54 | microgram |
| Glycine: | 69 | mg |
| Methionine: | 69 | mg |
| Sucrose: | 630 | mg |
| PEG: | 100 | mg |
| PVP: | 100 | mg |

10. The composition as claimed in claim 1 which is provided in the form of a unit dose to be reconstituted in 20 cl of water and which comprises:

| | | |
|---|---|---|
| Sodium bicarbonate: | 624.316 | mg |
| NaCl: | 317.400 | mg |
| KCl: | 212.010 | mg |
| Citric acid: | 356.696 | mg |
| Vitamin B1: | 0.32 | mg |
| Vitamin B6: | 0.59 | mg |
| Vitamin B9: | 133 | micrograms |
| Vitamin B12: | 1 | microgram |
| Glycine: | 126 | mg |
| Lactose: | 368.280 | mg |
| Dextrose: | 570 | mg |
| Gum acacia (fiber gum): | 408 | mg |

11. The composition as claimed in claim 1, for the rehydration of elderly people.

12. The composition as claimed in claim 1 wherein the glucose source is selected from the group consisting of glucose, dextrose, disaccharides and polysaccharides comprising at least one glucose subunit, selected from the group consisting of sucrose, lactose, starch, maltose, cellobiose, trehalose and mixtures thereof.

13. The composition as claimed in claim 2 wherein the glucose source is selected from the group consisting of glucose, dextrose, disaccharides and polysaccharides comprising at least one glucose subunit.

14. The composition as claimed in claim 2 wherein the water-soluble amino compound is selected from the group consisting of methionine, glutamine and glycine.

15. The composition as claimed in claim 2 wherein the sodium is provided by a compound selected from the group consisting of sodium chloride, sodium bicarbonate, sodium acetate, sodium hydrogen carbonate and sodium carbonate.

16. The composition as claimed in claim 9 for the rehydration of elderly people.

17. The composition as claimed in claim 10 for the rehydration of elderly people.

18. A method of preparing a solution comprising
adding to water a composition providing quantities relative to one liter of final composition of the following components:
35 to 75 mmol per liter of sodium
5 to 15 mmol per liter of potassium
11 to 50 mmol per liter of chloride
1 to 5 grams per liter of a source of glucose-providing carbohydrate
0.1 to 5 grams per liter of at least one water-soluble amino compound selected from the group consisting of amino acids, peptides and polypeptides;
at least one vitamin selected from the group consisting of:
vitamin C, in a quantity of 25 mg/l to 1 g/l
vitamin B1, in a quantity of 0.1 mg/l to 2.6 mg/l
vitamin B2, in a quantity of 0.2 mg/l to 3.2 mg/l
vitamin B3, in a quantity of 1.5 mg/l to 28 mg/l
vitamin B6, in a quantity of 0.15 mg/l to 4.4 mg/l
vitamin B8, in a quantity of 3 μg/l to 120 μg/l
vitamin B9, in a quantity of 30 μg/l to 2000 μg/l
vitamin B12, in a quantity of 0.5 μg/l to 20 μg/l
said solution having an osmolarity comprised between 150 and 200 mosmol/l.
